(19) United States
(12) Patent Application Publication
(10) Pub. No.: US 2023/0024745 A1
(43) Pub. Date: Jan. 26, 2023

(54) ESTERIFICATION OF ACETIC ACID RECOVERED FROM WOOD ACETYLATION WITH ETHER-ALCOHOLS

(71) Applicant: EASTMAN CHEMICAL COMPANY, KINGSPORT, TN (US)

(72) Inventors: MESFIN EJERSSA JANKA, KINGSPORT, TN (US); KEVIN JOHN FONTENOT, JONESBOROUGH, TN (US)

(73) Assignee: EASTMAN CHEMICAL COMPANY, KINGSPORT, TN (US)

(21) Appl. No.: 17/785,470

(22) PCT Filed: Dec. 16, 2019
(86) PCT No.: PCT/US2019/066470

§ 371 Date: Jun. 15, 2022

(51) Int. Cl. C07C 67/08; C07C 69/12; C07C 41/01

(52) U.S. Cl. CPC C07C 67/08; C07C 69/12; C07C 41/01

(57) ABSTRACT

An esterification process that uses an acetic acid composition from a wood acetylation process as a reactant. Even though the acetic acid composition contains impurities, such as ethyl acetate, methyl acetate, acetaldehyde, acetone, terpenes and/or terpenes derivatives, the impurities do not adversely affect the quality of the ether-ester products. This process can be economically advantageous by using cheaper acetic acid—one sourced directly from a wood acetylation process.

### TECHNICAL FIELD

The invention generally relates to the field of organic chemistry. It particularly relates to an esterification process that uses acetic acid from a wood acetylation process without adversely affecting the quality of the ether-ester products.

### BACKGROUND

Lignocellulosic material (e.g., wood) can undergo esterification (e.g., acetylation) to extend its service life by improving its resistance to weather and pathogens. In a typical wood acetylation process, the lignocellulosic material is contacted with acetic anhydride to acetylate the hydroxyl groups in the lignocellulosic material as described, for example, in WO 2005/077626. During this process, a byproduct stream containing acetic acid, acetic anhydride, ethyl acetate, methyl acetate, acetaldehyde, acetone, terpenes and/or terpenes derivatives is generated. This stream is typically subjected to one or more separation/purification steps before its contents are recycled, discarded, and/or used in another process.

The economic success of wood acetylation using acetic anhydride, however, can be greatly improved if the acetic acid-containing stream can be used without further separation/purification.

Thus, there is a need in the art to provide a process for using the acetic acid-containing byproduct stream from a wood acetylation process without the need to purify the stream.

The present invention addresses this need as well as others, which will become apparent from the following description and the appended claims.

### SUMMARY

The invention is as set forth in the appended claims.

Briefly, the invention provides a process for preparing an ether-ester.

In various embodiments, the process comprises esterifying a composition comprising acetic acid (AA) with an ether-alcohol in the presence of an acid catalyst to form an ether-ester. The AA composition comprises an impurity in an amount of at least 100 ppm, based on the total weight of the AA composition. The impurity comprises ethyl acetate, methyl acetate, acetaldehyde, acetone, terpenes, terpenes derivatives, or mixtures thereof.

In various other embodiments, the process comprises (a) acetylating wood with acetic anhydride to form an acetylated wood and an effluent comprising acetic acid and an impurity, and (b) esterifying the acetic acid in the effluent with an ether-alcohol in the presence of an acid catalyst and the impurity to form an acetate ester. The impurity comprises terpenes, terpenes derivatives, or mixtures thereof.

### DETAILED DESCRIPTION

It has been surprisingly discovered that a byproduct stream containing acetic acid generated in a wood acetylation process, without prior purification, can be reacted with ether-alcohols to make ether-esters without affecting the quality of the ether-ester products. That is, the ether-ester products made with the byproduct acetic acid can meet the same purity specifications as a product made using purified acetic acid without undergoing additional purification steps and/or without requiring more aggressive purification conditions than those employed for a product made with purified acetic acid.

Thus, the invention provides a process for preparing an ether-ester.

In one embodiment, the process comprises esterifying a composition comprising acetic acid (AA) with an ether-alcohol in the presence of an acid catalyst to form an ether-ester. The AA composition comprises an impurity in an amount of at least 100 ppm, based on the total weight of the AA composition, where the impurity comprises or are selected from ethyl acetate, methyl acetate, acetaldehyde, acetone, terpenes, terpenes derivatives, or mixtures thereof.

In various instances, the AA composition comprises an effluent from a wood acetylation process.

In various instances, the effluent from the wood acetylation process has not undergone purification before the esterification step.

In various instances, the esterification process produces the ether-ester at least at the same yield compared to a process where the effluent has undergone purification before the esterification step.

The amount of acetic acid in the AA composition that originates from a wood acetylation process is not particularly limiting. For example, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or even 100 wt % of the acetic acid in the AA composition can originate from a wood acetylation process, based on the total weight of acetic acid in the AA composition.

Other amounts of acetic acid in the AA composition that can originate from a wood acetylation process include 5 to 100 wt %, 10 to 100 wt %, 20 to 100 wt %, 30 to 100 wt %, 40 to 100 wt %, 50 to 100 wt %, 60 to 100 wt %, 70 to 100 wt %, 80 to 100 wt %, 90 to 100 wt %, 95 to 100 wt %, 5 to 90 wt %, 10 to 90 wt %, 20 to 90 wt %, 30 to 90 wt %, 40 to 90 wt %, 50 to 90 wt %, 60 to 90 wt %, 70 to 90 wt %, 80 to 90 wt %, 5 to 80 wt %, 10 to 80 wt %, 20 to 80 wt %, 30 to 80 wt %, 40 to 80 wt %, 50 to 80 wt %, 60 to 80 wt %, 70 to 80 wt %, 5 to 70 wt %, 10 to 70 wt %, 20 to 70 wt %, 30 to 70 wt %, 40 to 70 wt %, 50 to 70 wt %, 60 to 70 wt %, 5 to 60 wt %, 10 to 60 wt %, 20 to 60 wt %, 30 to 60 wt %, 40 to 60 wt %, 50 to 60 wt %, 5 to 50 wt %, 10 to 50 wt %, 20 to 50 wt %, 30 to 50 wt %, 40 to 50 wt %, 5 to 40 wt %, 10 to 40 wt %, 20 to 40 wt %, 30 to 40 wt %, 5 to 30 wt %, 10 to 30 wt %, 20 to 30 wt %, 5 to 20 wt %, or 10 to 20 wt %, based on the total weight of acetic acid in the AA composition.

In addition to acetic acid, the AA composition may comprise acetic anhydride. The acetic anhydride may take part in the esterification reaction, so its amount in the AA composition is not particularly limiting. For example, the AA composition may comprise up to 40 wt %, up to 35 wt %, up to 30 wt %, up to 25 wt %, up to 20 wt %, up to 15 wt %, up to 10 wt %, or up to 5 wt % of acetic anhydride, based on the total weight of the AA composition.

In various instances, the AA composition can comprise at least 0.01 wt %, at least 0.05 wt %, at least 0.1 wt %, at least 0.5 wt %, or at least 1 wt %, and in each case, up to 40 wt %, up to 35 wt %, up to 30 wt %, up to 25 wt %, up to 20 wt %, up to 15 wt %, up to 10 wt %, or up to 5 wt % of acetic anhydride, based on the total weight of the AA composition.

In various other instances, the AA composition can comprise from 5 to 40 wt %, 5 to 35 wt %, 5 to 30 wt %, 5 to 25 wt %, 5 to 20 wt %, 5 to 15 wt %, 5 to 10 wt %, 10 to 40 wt %, 10 to 35 wt %, 10 to 30 wt %, 10 to 25 wt %, 10 to 20 wt %, 10 to 15 wt %, 15 to 40 wt %, 15 to 35 wt %, 15 to 30 wt %, 15 to 25 wt %, or 15 to 20 wt % of acetic anhydride, based on the total weight of the AA composition.

In addition to acetic acid and optionally acetic anhydride, the AA composition comprises an impurity in an amount of at least 100 ppm, based on the total weight of the AA composition, where the impurity comprises or are selected from ethyl acetate, methyl acetate, acetaldehyde, acetone, terpenes, terpenes derivatives, or mixtures thereof.

By “terpenes derivatives,” it is meant one or more terpenes reaction products formed during a wood acetylation process, such as isobornyl acetate.

In various instances, the esterification process can produce the ether-ester at least at the same yield compared to a process using an AA composition comprising the impurity in an amount of less than 100 ppm.

The amount of impurity in the AA composition may be determined on an individual basis or in the aggregate. For example, in various instances, the total amount of all impurities in the AA composition is at least 100 ppm, based on the total weight of the AA composition. In various other instances, the AA composition contains at least one impurity in an amount of at least 100 ppm, based on the total weight of the AA composition.

The amount of impurity (either individually or in the aggregate) in the AA composition can be at least 100 ppm, at least 200 ppm, at least 300 ppm, at least 400 ppm, at least 500 ppm, at least 600 ppm, at least 700 ppm, at least 800 ppm, at least 900 ppm, or at least 1,000 ppm and in each case, up to 10,000 ppm, 9,000 ppm, 8,000 ppm, 7,000 ppm, 6,000 ppm, 5,000 ppm, 4,000 ppm, 3,000 ppm, or 2,000 ppm, based on the total weight of the AA composition.

An unexpected result of employing an acetic acid-containing composition from the acetylation of a lignocellulosic material in esterification reactions with an ether-alcohol concerns the presence of terpenes. Ordinarily, the presence of terpenes can render an acid composition unsuitable for use in processes that employ the acid as a reactant. That is because such processes can cause the terpenes to convert into tar or other substances, which can foul the process equipment used to carry out the reaction, or the terpenes can otherwise interfere with the desired reaction. However, it has been surprisingly discovered that the terpenes do not form species in amounts that can cause damage to the process equipment or otherwise interfere with the esterification reaction. Thus, while an acetic acid-containing composition from the acetylation of a lignocellulosic material may not be suitable in other processes, it is well suited for use as a source of acetic acid in esterification reactions with an ether-alcohol to form ether-esters.

Thus, in various instances, the impurity comprises terpenes, terpenes derivatives, or mixtures thereof.

The AA composition can comprises from 100 ppm, 500 ppm, or 1,000 ppm and in each case, up to 5,000 ppm, up to 4,000 ppm, up to 3,000 ppm, or 2,000 ppm of terpenes, terpenes derivatives, or mixtures thereof, based on the total weight of the AA composition.

The terpenes or terpenes derivatives may comprise α-pinene, camphene, limonene, ρ-cymene, γ-terpinene, α-terpinolene, isobornyl acetate, or mixtures thereof. The structures of these compounds are shown below.

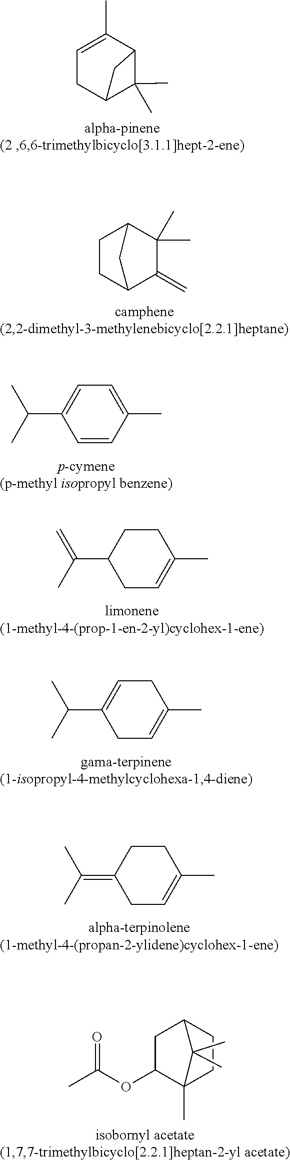

In various instances, the AA composition comprises limonene, pinene, or mixtures thereof.

There is no restriction on the ether-alcohol that can be used in the process of the invention. Typically, the ether-alcohol has the general formula (1):

R—(OCH₂CH(R¹))ₙ—OH (1)

and the ether-ester product has the general formula (2):

R—(OCH₂CH(R¹))ₙ—OC(O)CH₃ (2)

where R is an alkyl or aryl group having 1 to 20 carbon atoms; R¹ is hydrogen or methyl; n is 1 or 2; and when n is 2, R¹ is hydrogen.

In various instances, the alkyl or aryl group represented by R may have 1 to 18 carbon atoms, 1 to 16 carbon atoms, 1 to 14 carbon atoms, 1 to 12 carbon atoms, 1 to 10 carbon atoms, 1 to 8 carbon atoms, 1 to 6 carbon atoms, 1 to 5 carbon atoms, 1 to 4 carbon atoms, 1 to 3 carbon atoms, or 1 to 2 carbon atoms.

Specific examples of R groups include methyl, ethyl, propyl, iso-propyl, n-butyl, iso-butyl, n-pentyl, tert-pentyl, neopentyl, isopentyl, sec-pentyl, 3-pentyl, sec-isopentyl, 2-methylbutyl, n-hexyl, 2-methylpentyl, 3-methylpentyl, 2,3-dimethylbutyl, 2,2-dimethlybutyl, n-heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, n-octyl, 2-methylheptyl, 3-methylheptyl, 4-methylheptane, 2-ethylhexyl, 3-ethylhexyl, 2,2-dimethylhexyl, 2,3-dimethylhexyl, 2,4-dimethylhexyl, 2,5-dimethylhexyl, 3,3-dimethylhexyl, 3,4-dimethylhexyl, 3-ethyl-2-methylpentyl, 3-ethyl-3-methylpentyl, 2,2,3-trimethylpentyl, 2,2,4-trimethylpentyl, 2,3,3-trimethylpentyl, 2,3,4-trimethylpentyl, 2,2,3,3-tetramethylbutyl, phenyl, or benzyl group.

The starting ether-alcohol can be a single ether-alcohol or a mixture of ether-alcohols. In the latter case, a mixture of ether-esters can be formed.

The acid catalyst for the esterification reaction may be any known in the art useful for esterifying carboxylic acids with ether-alcohols. Examples of such catalysts include sulfuric acid, titanium sulfate, heteropolyacids, tungstophosphoric acid, solid acid catalysts, an alkyl sulfonic acid of the formula R′SO₃H where R′ represents a C₁ to C₁₂ substituted or unsubstituted aliphatic hydrocarbonyl group, or an alkyl benzene sulfonic acid of the formula R″C₆H₄SO₃H where R″ represents an alkyl radical having from 1 to 20 carbon atoms.

The esterification reaction may be carried out at any suitable reaction temperature and pressure. For instance, it may be performed at pressures ranging from atmospheric pressure to 500 psig. Likewise, the esterification reaction may generally be conducted at a temperature ranging from 50° C. to 200° C.

In various instances, the esterification step is conducted within 20 miles, 15 miles, 10 miles, 5 miles, 3 miles, or 1 mile of a wood acetylation process.

Esterification of acetic acid (all or a portion of it originating from a wood acetylation process) with an ether-alcohol in presence of an acid catalyst produces an equilibrium esterification reaction mixture that contains ether-ester, water, unreacted alcohol, unreacted acetic acid, and impurities. Purification of the ether-ester product and recovery of the unreacted acetic acid and unreacted alcohol can be achieved utilizing one or more distillation columns, decanters, or any other purification techniques known in the art.

In various instances, the esterification process further comprises the steps of (a) acetylating wood with acetic anhydride to form an acetylated wood and an effluent comprising acetic acid and the impurity, and (b) passing at least a portion of the effluent to the esterification step without first purifying the effluent.

In another embodiment, the process for preparing an ether-ester comprises the steps of (a) acetylating wood with acetic anhydride to form an acetylated wood and an effluent comprising acetic acid and an impurity, and (b) esterifying the acetic acid in the effluent with an ether-alcohol in the presence of an acid catalyst and the impurity to form an ether-ester where the impurity comprises terpenes, terpenes derivatives, or mixtures thereof.

The form of the wood suitable for use in the acetylation step is not limiting and can be any shape or dimension. For example, the wood can be in the form of veneers, boards, planks, squared timber, beams or profiles, wood particles, wood flakes, or wooden end-products. When wood particles are employed, the wood scrap can be in the form of wood flour, wood fibers, and wood shavings obtained from wood processing. Mixtures of wood scraps can also be used. Additionally, the species of wood is not limiting, as any species of wood can be employed. In some instances, the wood can comprise broad-leaved or coniferous wood (generally speaking, hard or soft woods, respectively).

The wood can contain water. In some instances, the wood can initially contain at least 15 wt %, at least 17 wt %, or at least 19 wt % of water prior to acetylation. In some instances, the wood can be dewatered to produce a dewatered material having a water content of less than 15 wt %, less than 10 wt %, or less than 5 wt % of water. Any method known in the art can be employed to achieve the desired water content prior to acetylation. In some instances, kiln drying and/or drying by acetic acid impregnation coupled with vacuum/pressure cycles can be employed to achieve the desired water content.

The acetic anhydride can be employed in any amount sufficient to increase the total acetyl content of the wood by at least 1 wt %, at least 2 wt %, or at least 3 wt %, based on the total weight of the wood. The total acetyl content of the wood can be determined according to the saponification method, as is known in the art. In some instances, the amount of acetic anhydride absorbed by the wood can be in the range of 50 to 250 wt %, 65 to 200 wt %, or 80 to 150 wt %, based on the weight of the dewatered wood.

The acetylation step is typically performed at elevated pressure and/or temperature. The pressure and temperature employed can vary, depending on the desired increase in the total acetyl content of the wood. In some instances, the acetylation can be performed at temperatures of at least 40° C., at least 65° C., or at least 90° C. Additionally, the acetylation can be performed at a pressure of at least 20 psig, such as from 25 to 150 psig, from 35 to 125 psig, or from 50 to 100 psig.

Following acetylation, the wood can be subject to a drying step so as to remove any excess acetic anhydride and residual acid remaining in the wood. In some instances, the drying step can be performed in the same reaction vessel as the acetylation step. The drying step can be any known in the art capable of lowering the free acid content of the acetylated wood to any desired level. Examples of drying techniques that can be employed include applying heat with an inert gas (e.g., nitrogen) flow, adding steam to the reaction vessel, and/or drying in a kiln which can be equipped to collect any acid removed via condensation.

The acetylation of wood with acetic anhydride produces acetylated wood and acetic acid as a byproduct. In some instances, at least a portion of the acetic acid produced during the acetylation step can be recycled and reused.

However, in some cases, at least a portion of the acetic acid originating in the wood acetylation step can be removed from the acetylation reactor. This prevents contaminants drawn from the wood from building up in the system. For example, terpenes from the wood can become entrained with the acetic acid resulting from the acetylation. Accordingly, at least a portion of the acid-containing composition can be removed from the system as a non-recycled, acid-containing composition.

The amount of the acid-containing composition withdrawn from the acetylation system can range from 0.01 to 25 wt %, from 0.05 to 15 wt %, or from 0.1 to 5 wt % of the total amount of the acid-containing composition withdrawn from acetylation reactor.

In accordance with the invention, at least a portion of the acid-containing composition originating from the acetylation of wood can be employed in a process for making the ether-ester.

In various instances, the acid-containing composition/effluent from the acetylation of wood step has not undergone purification before the esterification step.

In various instances, the esterification process produces the ether-ester at least at the same yield compared to a process where the effluent has undergone purification before the esterification step.

In various instances, the effluent comprises from 100 to 5,000 ppm of terpenes, terpenes derivatives, or mixtures thereof.

In various instances, the terpenes or terpenes derivatives comprise α-pinene, camphene, limonene, ρ-cymene, γ-terpinene, α-terpinolene, isobornyl acetate, or mixtures thereof.

In various instances, the terpenes comprise limonene, pinene, or mixtures thereof.

In various instances, the effluent comprises up to 40 wt %, up to 35 wt %, up to 30 wt %, up to 25 wt %, up to 20 wt %, up to 15 wt %, up to 10 wt %, up to 5 wt %, 5 to 40 wt %, 5 to 35 wt %, 5 to 30 wt %, 5 to 25 wt %, 5 to 20 wt %, 5 to 15 wt %, 5 to 10 wt %, 10 to 40 wt %, 10 to 35 wt %, 10 to 30 wt %, 10 to 25 wt %, 10 to 20 wt %, 10 to 15 wt %, 15 to 40 wt %, 15 to 35 wt %, 15 to 30 wt %, 15 to 25 wt %, or 15 to 20 wt % of acetic anhydride, based on the total weight of the effluent/AA composition.

The description of the esterification step in the first embodiment applies equally to this embodiment.

To remove any doubt, the present invention includes and expressly contemplates and discloses any and all combinations of embodiments, features, characteristics, parameters, and/or ranges mentioned herein. That is, the subject matter of the present invention may be defined by any combination of embodiments, features, characteristics, parameters, and/or ranges mentioned herein.

It is contemplated that any ingredient, component, or step that is not specifically named or identified as part of the present invention may be explicitly excluded.

Any process/method, apparatus, compound, composition, embodiment, or component of the present invention may be modified by the transitional terms “comprising,” “consisting essentially of,” or “consisting of,” or variations of those terms.

As used herein, the indefinite articles “a” and “an” mean one or more, unless the context clearly suggests otherwise. Similarly, the singular form of nouns includes their plural form, and vice versa, unless the context clearly suggests otherwise.

While attempts have been made to be precise, the numerical values and ranges described herein should be considered as approximations, unless the context indicates otherwise. These values and ranges may vary from their stated numbers depending upon the desired properties sought to be obtained by the present disclosure as well as the variations resulting from the standard deviation found in the measuring techniques. Moreover, the ranges described herein are intended and specifically contemplated to include all sub-ranges and values within the stated ranges. For example, a range of 50 to 100 is intended to include all values within the range including sub-ranges such as 60 to 90, 70 to 80, etc.

Any two numbers of the same property or parameter reported in the working examples may define a range. Those numbers may be rounded off to the nearest thousandth, hundredth, tenth, whole number, ten, hundred, or thousand to define the range.

The content of all documents cited herein, including patents as well as non-patent literature, is hereby incorporated by reference in their entirety. To the extent that any incorporated subject matter contradicts with any disclosure herein, the disclosure herein shall take precedence over the incorporated content.

This invention can be further illustrated by the following working examples, although it should be understood that these examples are included merely for purposes of illustration and are not intended to limit the scope of the invention.

### EXAMPLES

Analytical Methods

Gas Chromatographic Mass Selective Method 1

Samples were analyzed using a Thermo Scientific DSQ Single Quad Mass Spectrometer with Trace Ultra Gas Chromatograph and a Tri-Plus Autosampler for liquid injections (or equivalent).

The gas chromatograph was equipped with a split/heated injector (250° C.) and a capillary column (30 meter×0.25 mm ID) coated with (50% phenyl)-methylpolysiloxane at 0.25 mm film thickness (such as DB-17 equivalent). Helium was used as the carrier gas at a constant flow of 1.5 mL/minute, calculated and programmed within the gas chromatograph.

The column temperature was programmed as follows: The initial oven temperature was set at 40° C. and held for 1 minute, the oven was ramped up to 150° C. at 6° C./minute and was held at 150° C. for 5 minutes, then the oven was ramped up to 300° C. at 20° C./minute and was held at 300° C. for 5 minutes (the total run time was 36 minutes).

1.0 μL of the prepared sample solution was injected with a split ratio of 7:1. Thermo Xcalibur Quant data system software was used for data acquisition and processing. The single quad mass spectrometer was set with a source temperature of 250° C., a gain of 3, and a MS transfer line temperature of 280° C.

An internal standard solution was prepared by dissolving 1 μL of p-dichlorobenzene in 1.0 mL of glacial acetic acid.

Samples were prepared by pipetting 1.0 mL of each sample into a vial, to which 3 μL of the internal standard solution was added with a syringe (701 N Hamilton, or equivalent). The Thermo DSQ mass spectrometer was set to monitor selected/specific masses: m/z=91 (p-Cymene), m/z=93 (dl-Limonene, γ-Terpinene, and Terpinolene), m/z=136 (α-Pinene, Camphene), and m/z=146 (p-Dichlorobenzene). Positive identifications in samples of interest were made using selective mass detection, as well as retention time comparison with known standards.

Calibration standards were prepared using reference material purchased from Aldrich (at 95% purity or better) in the following way: A stock solution was prepared by adding 0.0244 g γ-terpinene, 0.0216 g α-pinene, 0.0231 g p-cymene, 0.0259 g terpinolene, 0.0264 g dl-limonene, and 0.0266 g camphene to a 100 mL volumetric flask where the volume was brought to 100 mL with glacial acetic acid. Five calibration standards were prepared. See Table 1.

TABLE 1

| | | 1:1000 | | | | |
|---|---|---|---|---|---|---|
| | | dilution | | | | |
| | | (1:10 | | | | |
| | | dilution of | 0.5 mL | 1 mL | 1 mL | 1 mL |
| | Weight/ | standard | stock/ | stock/ | stock/ | stock/ |
| | 100 mL | 3) | 100 mL | 100 mL | 50 mL | 25 mL |
| | Stock | Standard | Standard | Standard | Standard | Standard |
| Component | Solution | 1 | 2 | 3 | 4 | 5 |
| γ-Terpinene | 0.0244 g | 0.24 ppm | 1.2 ppm | 2.4 ppm | 4.9 ppm | 9.8 ppm |
| α-Pinene | 0.0216 g | 0.22 ppm | 1.1 ppm | 2.2 ppm | 4.3 ppm | 8.6 ppm |
| p-Cymene | 0.0231 g | 0.23 ppm | 1.2 ppm | 2.3 ppm | 4.6 ppm | 9.2 ppm |
| Terpinolene | 0.0259 g | 0.26 ppm | 1.3 ppm | 2.6 ppm | 5.2 ppm | 10.4 ppm |
| D-Limonene | 0.0264 g | 0.26 ppm | 1.3 ppm | 2.6 ppm | 5.3 ppm | 10.6 ppm |
| Camphene | 0.0266 g | 0.27 ppm | 1.3 ppm | 2.7 ppm | 5.3 ppm | 10.6 ppm |

Standard 5 was prepared by diluting 1.0 mL of the stock solution into 25 mL of glacial acetic acid, volumetrically. 1.0 mL of Standard 5 was placed in a vial, to which 3.0 μL of the internal standard solution was added.

Standard 4 was prepared by diluting 1.0 mL of the stock solution into 50 mL of glacial acetic acid, volumetrically. 1.0 mL of Standard 4 was placed in a vial, to which 3.0 μL of the internal standard solution was added.

Standard 3 was prepared by diluting 1.0 mL of the stock solution into 100 mL of glacial acetic acid, volumetrically. 1.0 mL of Standard 3 was placed in a vial, to which 3.0 μL of the internal standard solution was added.

Standard 2 was prepared by diluting 0.5 mL of the stock solution into 100 mL of glacial acetic acid, volumetrically. 1.0 mL of Standard 2 was placed in a vial, to which 3.0 μL of the internal standard solution was added.

Standard 1 was prepared by diluting 1.0 mL of Standard 3 into 10 mL of glacial acetic acid, volumetrically. 1.0 mL of Standard 1 was placed in a vial, to which 3.0 μL of the internal standard solution was added.

These five calibration standards (with internal standard added) were used in the approximate range of 0.2 ppm to 11 ppm for each of γ-terpinene, α-pinene, p-cymene, terpinolene, dl-limonene, and camphene to make a 5-point linear calibration for each compound. The resulting linear calibration equation was calculated by the Xcalibur software and used by the data system to determine quantitative results in samples of interest.

Gas Chromatographic Mass Selective Method 2

Samples were analyzed using a Thermo Scientific DSQ Single Quad Mass Spectrometer with Trace Ultra Gas Chromatograph and a Tri-Plus

Autosampler for liquid injections (or equivalent).

The gas chromatograph was equipped with a split/heated injector (250° C.) and a capillary column (30 meter×0.25 mm ID) coated with polyethylene glycol at 0.25 mm film thickness (such as DB-WAX equivalent). Helium was used as the carrier gas at a constant flow of 1.5 mL/minute, calculated and programmed within the gas chromatograph.

The column temperature was programmed as follows: The initial oven temperature was set at 40° C. and held for 1 minute, the oven was ramped up to 150° C. at 8° C./minute and was held at 150° C. for 2 minutes, then the oven was ramped up to 240° C. at 20° C./minute and was held at 240° C. for 10 minutes (the total run time was 31 minutes).

1.0 μL of the prepared sample solution was injected with a split ratio of 7:1. Thermo Xcalibur Quant data system software was used for data acquisition and processing. The single quad mass spectrometer was set with a source temperature of 250° C., a gain of 3, and a MS transfer line temperature of 280° C.

An internal standard solution was prepared by dissolving 1 μL of p-dichlorobenzene in 1.0 mL of glacial acetic acid.

Samples were prepared by pipetting 1.0 mL of each sample into a vial, to which 3 μL of the internal standard solution was added with a syringe (701 N Hamilton, or equivalent). The Thermo DSQ mass spectrometer was set to monitor selected/specific masses: m/z=95 (Isobornyl Acetate), and m/z=146 (p-Dichlorobenzene). Positive identifications in samples of interest were made using selective mass detection, as well as retention time comparison with known standards.

Calibration standards were prepared using reference material purchased from Aldrich (at 95% purity or better) in the following way: A stock solution was prepared by adding 0.0259 g isobornyl acetate to a 100 mL volumetric flask where the volume was brought to 100 mL with glacial acetic acid. Five calibration standards were prepared. See Table 2.

TABLE 2

| | | 1:1000 | | | | |
|---|---|---|---|---|---|---|
| | | dilution | | | | |
| | | (1:10 | | | | |
| | | dilution of | 0.5 mL | 1 mL | 1 mL | 1 mL |
| | Weight/ | standard | stock/ | stock/ | stock/ | stock/ |
| | 100 mL | 3) | 100 mL | 100 mL | 50 mL | 25 mL |
| | Stock | Standard | Standard | Standard | Standard | Standard |
| Component | Solution | 1 | 2 | 3 | 4 | 5 |
| Isobornyl | 0.0259 g | 0.26 ppm | 1.3 ppm | 2.6 ppm | 5.2 ppm | 10.4 ppm |
| Acetate | | | | | | |

Standard 5 was prepared by diluting 1.0 mL of the stock solution into 25 mL of glacial acetic acid, volumetrically. 1.0 mL of Standard 5 was placed in a vial, to which 3.0 μL of the internal standard solution was added.

Standard 4 was prepared by diluting 1.0 mL of the stock solution into 50 mL of glacial acetic acid, volumetrically. 1.0 mL of Standard 4 was placed in a vial, to which 3.0 μL of the internal standard solution was added.

Standard 3 was prepared by diluting 1.0 mL of the stock solution into 100 mL of glacial acetic acid, volumetrically. 1.0 mL of Standard 3 was placed in a vial, to which 3.0 μL of the internal standard solution was added.

Standard 2 was prepared by diluting 0.5 mL of the stock solution into 100 mL of glacial acetic acid, volumetrically. 1.0 mL of Standard 2 was placed in a vial, to which 3.0 μL of the internal standard solution was added.

Standard 1 was prepared by diluting 1.0 mL of Standard 3 into 10 mL of glacial acetic acid, volumetrically. 1.0 mL of Standard 1 was placed in a vial, to which 3.0 μL of the internal standard solution was added.

These five calibration standards (with internal standard added) were used in the approximate range of 0.2 ppm to 11 ppm of isobornyl acetate to make a 5-point linear calibration for the compound. The resulting linear calibration equation was calculated by the Xcalibur software and used by the data system to determine quantitative results in samples of interest.

### Example 1

GC Method 1 was used to determine the amount of terpenes impurities (α-pinene, camphene, limonene, ρ-cymene, γ-terpinene, and α-terpinolene) in an effluent stream containing acetic acid from a wood acetylation process. The results are reported in Table 3.

GC Method 2 was used to determine the amount of a terpene derivative (isobornyl acetate) in an effluent stream containing acetic acid from a wood acetylation process. The result is reported in Table 3.

TABLE 3

| Species of Terpenes or Terpene | |
|---|---|
| Derivative | Amount |
| in Wood Acetylation Effluent | (ppm) |
| α-pinene | 35.6 |
| camphene | 43 |
| limonene | 164.3 |
| ρ-cymene | 30.8 |
| γ-terpinene | 9.12 |
| α-terpinolene | 33 |
| isobornyl acetate* | 0.99 |

*Possible isomer peak was detected.

The invention has been described in detail with particular reference to specific embodiments thereof, but it will be understood that variations and modifications can be made within the spirit and scope of the invention.

1. A process for preparing an ether-ester, the process comprising:
esterifying a composition comprising acetic acid (AA) with an ether-alcohol in the presence of an acid catalyst to form an ether-ester,
wherein the AA composition comprises an impurity in an amount of at least 100 ppm, based on the total weight of the AA composition,
wherein the impurity comprises ethyl acetate, methyl acetate, acetaldehyde, acetone, terpenes, terpenes derivatives, or mixtures thereof.

2. The process according to claim 1, wherein the AA composition comprises an effluent from a wood acetylation process.

3. The process according to claim 2, wherein the effluent has not undergone purification before the esterification step.

4. The process according to claim 3, which produces the ether-ester at least at the same yield compared to a process where the effluent has undergone purification before the esterification step.

5. The process according to claim 1, wherein the impurity comprises terpenes, terpenes derivatives, or mixtures thereof.

6. The process according to claim 1, wherein the AA composition comprises 100 to 5,000 ppm of terpenes, terpenes derivatives, or mixtures thereof.

7. The process according to claim 1, wherein the terpenes or terpenes derivatives comprise α-pinene, camphene, limonene, ρ-cymene, γ-terpinene, α-terpinolene, isobornyl acetate, or mixtures thereof.

8. The process according to claim 1, wherein the AA composition comprises limonene, pinene, or mixtures thereof.

9. The process according to claim 1, wherein 5 to 100 wt of the acetic acid in the AA composition originates from a wood acetylation process.

10. The process according to claim 1, wherein the ether-alcohol has the general formula (1):
R—(OCH₂CH(R¹))ₙ—OH (1)
and the ether-ester has the general formula (2):
R—(OCH₂CH(R¹))ₙ—OC(O)CH₃ (2)
where
R is an alkyl or aryl group having 1 to 20 carbon atoms;
R¹ is hydrogen or methyl;
n is 1 or 2; and
when n is 2, R¹ is hydrogen.

11. The process according to claim 10, wherein R is a methyl, ethyl, propyl, iso-propyl, n-butyl, iso-butyl, n-pentyl, tert-pentyl, neopentyl, isopentyl, sec-pentyl, 3-pentyl, sec-isopentyl, 2-methylbutyl, n-hexyl, 2-methylpentyl, 3-methylpentyl, 2,3-dimethylbutyl, 2,2-dimethlybutyl, n-heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, n-octyl, 2-methylheptyl, 3-methylheptyl, 4-methylheptane, 2-ethylhexyl, 3-ethylhexyl, 2,2-dimethylhexyl, 2,3-dimethylhexyl, 2,4-dimethylhexyl, 2,5-dimethylhexyl, 3,3-dimethylhexyl, 3,4-dimethylhexyl, 3-ethyl-2-methylpentyl, 3-ethyl-3-methylpentyl, 2,2,3-trimethylpentyl, 2,2,4-trimethylpentyl, 2,3,3-trimethylpentyl, 2,3,4-trimethylpentyl, 2,2,3,3-tetramethylbutyl group, phenyl, or benzyl group.

12. The process according to claim 10, which produces the ether-ester at least at the same yield compared to a process using an AA composition comprising the impurity in an amount of less than 100 ppm.

13. The process according to claim 10, wherein the esterification step is conducted within 20 miles of a wood acetylation process.

14. The process according to claim 10, which further comprises:
acetylating wood with acetic anhydride to form an acetylated wood and an effluent comprising acetic acid and the impurity; and
passing at least a portion of the effluent to the esterification step without first purifying the effluent.

15. The process according to claim 10, wherein the AA composition comprises up to 40 wt % of acetic anhydride, based on the total weight of the AA composition.

16. A process for preparing an ether-ester, the process comprising:
acetylating wood with acetic anhydride to form an acetylated wood and an effluent comprising acetic acid and an impurity; and
esterifying the acetic acid in the effluent with an ether-alcohol in the presence of an acid catalyst and the impurity to form an ether-ester,
wherein the impurity comprises terpenes, terpenes derivatives, or mixtures thereof.

17. The process according to claim 16, wherein the effluent has not undergone purification before the esterification step.

18. The process according to claim 17, which produces the ether-ester at least at the same yield compared to a process where the effluent has undergone purification before the esterification step.

19. The process according to claim 16, wherein the effluent comprises 100 to 5,000 ppm of terpenes, terpenes derivatives, or mixtures thereof.

20. The process according to claim 16, wherein the terpenes or terpenes derivatives comprise α-pinene, camphene, limonene, ρ-cymene, α-terpinene, α-terpinolene, isobornyl acetate, or mixtures thereof.

21. (canceled)

22. (canceled)
